(19) United States
(12) Patent Application Publication
(10) Pub. No.: US 2017/0052234 A1
(43) Pub. Date: Feb. 23, 2017

(54) SIZE-ADJUSTABLE PROTECTING AND IMMOBILIZING SLEEVES FOR MRI DEVICES

(71) Applicant: ASPECT IMAGING LTD., Shoham (IL)

(72) Inventor: Uri RAPOPORT, Moshav Ben Shemen (IL)

(21) Appl. No.: 14/707,452

(22) Filed: May 8, 2015

Related U.S. Application Data
(60) Provisional application No. 61/992,272, filed on May 13, 2014.

(51) Int. Cl. G01R 33/28; G01R 33/30; G01R 33/38; A61B 5/055; A61B 5/00

(52) U.S. Cl. CPC G01R 33/288; A61B 5/0555; A61B 5/702; G01R 33/3806; G01R 33/30

(57) ABSTRACT

A method of radiofrequency (RF) protecting a body organ to be scanned by an open bore magnetic resonance imaging device (MRD) having a volume of interest for imaging a body portion and an inlet aperture for inserting the body portion within the bore. The method includes adjusting a size of a sleeve such as the sleeve is set tightly at least a portion of the organ to be scanned. The sleeve is an electrically earthed and size adjustable.

### FIELD OF THE INVENTION

The present invention pertains to size-adjustable protecting and immobilizing sleeves for MRI devices and to method thereof.

### BACKGROUND OF THE INVENTION

This invention relates to the field of magnetic resonance imaging (MRI), where radiofrequency (RF) magnetic fields are used to interrogate a region of interest. It is particularly directed towards the shielding of RF effects which occur from inside and outside of the MRI system.

One of the components in MRI systems is an RF coil, which is part of a typical MRI data gathering sequence. MRI RF signals of suitable frequencies are transmitted into the imaging volume and NMR responsive RF signals are then received from the imaging volume via one or more RF coils or antennae. Information encoded within the frequency and phase parameters of the received RF signals is then processed to form visual images representing the distribution of NMR nuclei within a cross-section or volume of the patient within the volume of interest (VOI) of the MRI system.

In MRI devices (MRDs) which are meant for imaging body extremities, only the portion to be examined in the MRI is located within the bore of the MRI system, whereas the rest of the body remains outside of the MRI. In such cases, RF signals from the volume outside the MRI may be collected through the conductive body and the extremity to be examined, having dielectric properties, may serve as a transceiver of RF signals, thus causing noise in signal collection during an MRI examination.

Additionally, RF fields within the MRI can induce an electrical current in the body which is transformed into energy in the form of heat. Heating of tissues is due to resistance in the tissues is called “ohmic heating”. Specific Absorption Rate (SAR) is key variable in determining patient heating potential in an MR scanner is the RF power absorbed by the body per unit mass, generally measured in the unit of W/kg. If the SAR exceeds the thermal regulation capacity the patient's body temperature will rise.

U.S. Pat. No. 5,304,932 is directed towards shielding an MRI RF coil from extraneous noise sources using an extremely thin conductive shield interposed between the RF coil and the static magnetic structure of an MRI system. To control eddy currents induced in such conductor by the changing magnetic flux of MRI gradient coils, the RF shield conductor thickness is less than three skin depths at the MRI RF operating frequencies of the RF coil. Preferably, the RF shield conductor thickness is on the order of only one skin depth or less

U.S. Pat. No. 7,801,613 is directed towards housing of implantable medical devices in a titanium alloy that provides improved electrical performance, mechanical strength, and reduced MRI heating.

There thus remains a long felt need for reducing the electromagnetic energy propagation from MRIs magnet bore to the outer environment surrounding the magnet and vice versa, for a protecting means providing effective Faraday shielding used as a barrier between the internal and external RF fields, and for reducing SAR of a non-examined portion of a body extremity in the vicinity of a MRD.

Subject motion and associated artifacts limit the applicability of magnetic resonance imaging (MRI) and the achievable quality of the images acquired, See Zaitsev et al., Magnetic resonance imaging of freely moving objects: prospective real-time motion correction using an external optical motion tracking system, NeuroImage 31(3), 1038-1050 (2006). Post-processing techniques have been developed to suppress MRI artifact arising from object planar rigid motion, See for example Zoroofi et al., IEEE Transaction on Medical Imaging, 15(6), 768-784 (1996). Moreover, a few patents, such as U.S. Pat. No. 5,602,891 discloses computerized tomography (CT) scanners & functional MRI (fMRI) imaging apparatus with means for compensation object motion.

As presented by Ranieri (2011), during fMRI acquisition, light restraints (i.e. foam wedges, vacuum pillows, straps, etc.) are used to help limit head motion. These restraints are most effective in restricting motion in the medial-lateral direction, and less effective for motion in orthogonal directions. With the desire to keep patient discomfort and stress at a minimum, head restraint is only lightly used and is not an extremely effective technique for preventing motion in fMRI, See https://tspace.library.utoronto.ca/bitstream/1807/29603/6/Ranieri_Shawn_M-201106_MHSc_thesis.pdf.

Both U.S. Pat. Appl. Pub. No. 2014009010 and U.S. Pat. Appl. Appl. No. 61/775,717 by Aspect Imaging Ltd are entirely incorporated herein as a reference.

Hence, MRI devices targeted for avoiding motion artifacts and specialized in producing an image sequence with reduced object-movement affect, and especially such as MRI systems adapted to image uncontrollably movable objects, such as neonates, premature babies and laboratory animals; and especially in those special cases were restrain is to be avoided, is still a long felt and unmet need.

### SUMMARY OF THE INVENTION

It is thus an object of the present invention to disclose, in an open bore MRD having a volume of interest for imaging a body portion and an inlet aperture for inserting said body portion within said bore, an electrically earthed size adjustable-size protecting and/or immobilizing sleeve.

It is another object of the present invention to disclose an MRI-proof adjustable-size protecting sleeve characterized by a telescopic elongation mechanism.

It is another object of the present invention to disclose an n inflatable MRI-proof adjustable-size protecting sleeve.

It is another object of the present invention to disclose an MRI-proof adjustable-size protecting sleeve comprising pressure applying mechanism.

It is another object of the present invention to disclose an MRI-proof adjustable protecting sleeve comprising one more sensors.

It is another object of the present invention to disclose a method of RF protecting and/or immobilizing a body organ to be scanned by an open bore MRD having a volume of interest for imaging a body portion and an inlet aperture for inserting said body portion within said bore, comprising step of size adjusting said a sleeve such as said sleeve is set tightly at least a portion of the organ to be scanned.

It is another object of the present invention to disclose a method of RF protecting and/or immobilizing a body organ to be scanned by an open bore MRD, comprising step of providing an MRI-proof adjustable-size protecting sleeve characterized by a telescopic elongation mechanism.

It is another object of the present invention to disclose a method of RF protecting and/or immobilizing a body organ to be scanned by an open bore MRD, comprising step of providing an inflatable MRI-proof adjustable-size protecting sleeve.

It is another object of the present invention to disclose a method of RF protecting and/or immobilizing a body organ to be scanned by an open bore MRD, comprising step of providing an MRI-proof adjustable-size protecting sleeve comprising pressure applying mechanism.

It is another object of the present invention to disclose a method of RF protecting and/or immobilizing a body organ to be scanned by an open bore MRD, comprising step of providing an MRI-proof adjustable protecting sleeve comprising one more sensors.

It is another object of the present invention to disclose a method as defined in any of the above, comprising a step of inflating or deflating said sleeve.

It is another object of the present invention to disclose a method as defined in any of the above, comprising a step of applying, by means of said sleeve, a pressure upon said organ to be scanned.

It is another object of the present invention to disclose a method as defined above, comprising step of regulating said pressure by either manual or automatic feed backed mechanism.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 schematically illustrates, in an out-of-scale manner, a front view of an open bore MRI device.

FIG. 2 schematically illustrates, in an out-of-scale manner, a set of three human right hands, a neonate hand (1a), a toddler hand (1b) and a 40 years old's hand (1c).

FIG. 3 schematically illustrates, in an out-of-scale manner, the aforesaid set of three human right hands (1a-c), accommodated within a size-adjustable sleeve (3a-c, respectively).

FIGS. 4a-b schematically illustrate, in an out-of-scale manner, a size-adjustable sleeve and the aforesaid set of three human right hands (1a-c), accommodated within a size-adjustable sleeve (40a-c, respectively).

FIG. 5 schematically illustrates, in an out-of-scale manner, a size-adjustable sleeve in its full elongated configuration, 40c.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAIL DESCRIPTION OF THE PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provides means, such as a protecting sleeve, and methods for reducing the electromagnetic energy propagation from an MRD's magnet-bore to the outer environment surrounding said magnet, and vice versa

The term “about” herein indicates a value within ±25%. The term “Faraday shield” refers hereinafter to an enclosure formed by a conducting material or by a mesh of such material, blocking external static and non-static electric fields. The term “magnetic fringe field” refers hereinafter to spatial gradient magnetic field surrounding the bore of said MRI in three orthogonal magnetic gradients which produces an attractive translational force on ferromagnetic objects. The magnetic in that zone is between 300-5 Gauss and is delimited by a 5-Gauss line. (“zone 4”). The term “RF” refers hereinafter to radiofrequency (RF) in the range of about 3 kHz to 300 GHz.

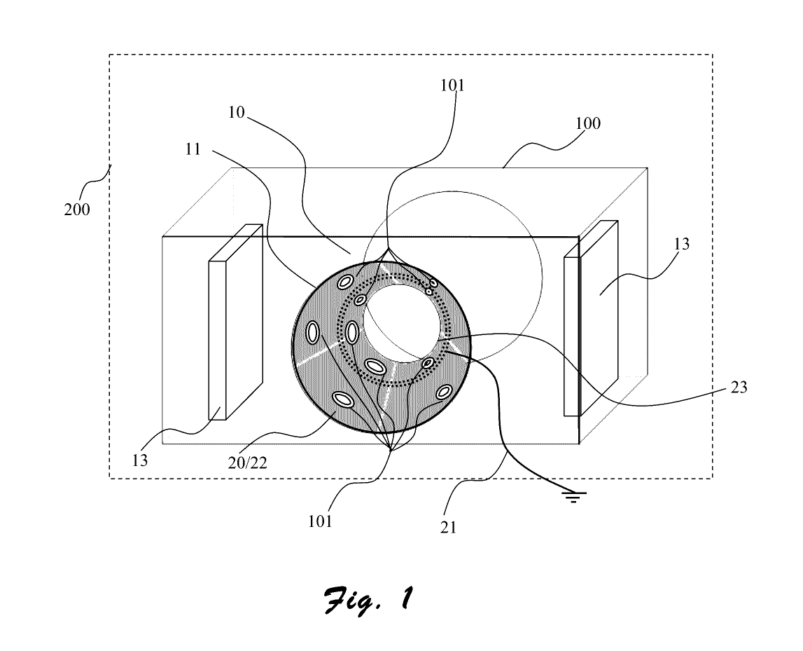

Reference is now made to FIG. 1 schematically illustrating in an out-of-scale manner a front view of an open bore MRI device (MRD, e.g., an MRI wrist system 100) according to one embodiment of the invention, comprising one or more magnets (here for example, permanent magnet 13, wherein superconductive) and a volume of interest for imaging a predefined body portion and an inlet aperture (11) for inserting a body portion within said bore (10), an electrically earthed (21) protecting sleeve (20), immobilizer, tourniquet bandage, or any other constricting or compressing device for reducing the electromagnetic energy propagation from said magnet bore (10) to the outer environment (200) surrounding MRD (100) and vice versa and/or for immobilizing said body portion. Sleeve (20) comprises a distal portion insertably locatable within bore (10) and a proximal portion (22) attachable to the MRD aperture (11), length and diameter of the sleeve is adapted to accept a non-imaged portion of a body portion insertable within bore (10) whilst the imaged portion protrudes from distal end of sleeve.

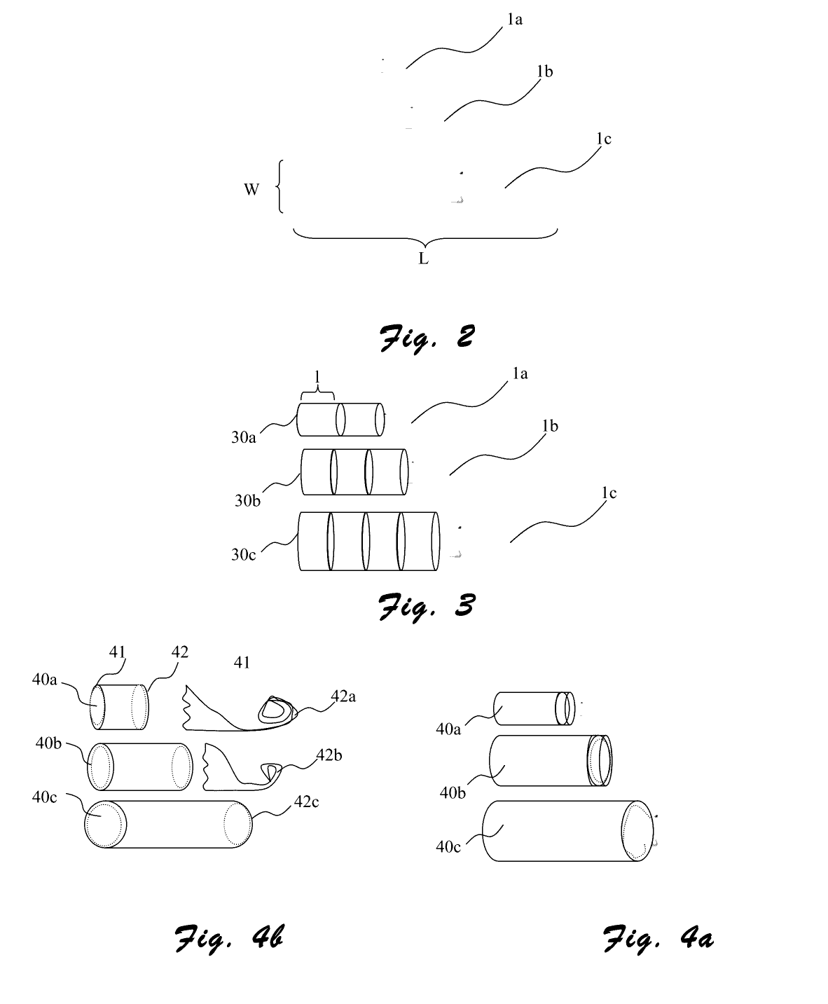

Reference is now made to FIG. 2, schematically illustrating an out-of-scale manner a set of three human right hands, a neonate hand (1a), a toddler hand (1b) and a 40 years old's hand (1c). Each hand has an arm (width W) and palm (total length palm+arm equals L). Thus, each hand (1a-c) is characterized by different L and W typical

Reference is now made to FIG. 3, schematically illustrating an out-of-scale manner the aforesaid set of three human right hands (1a-c), accommodated within a size-adjustable sleeve (3a-c, respectively). Hands are example for a body portion. Any other cylinderal portions (legs etc.) and non- or semi-cylinderal body parts can be easy accommodated within a sleeve disclosed herein or the like. In one embodiment of the technology presented in this invention, the sleeve comprises two or more inflatable or otherwise linearly reciprocated segments. Hence, in a relatively short hand (1a), only two segments (30a) are inflated, in a medium size hand three segments are inflated (30b) and for imaging a grown man's hand, four-segments are inflated (30c). The inflation is provided, e.g., by condensing an MRI-safe fluid such as air, or N₂, water etc. in other systems. This telescopic sleeve comprises two or more fluidly interconnected segments (air pockets or the like) each of which is surrounding the arm in a perpendicular manner in respect to arm's main longitudinal axis. The term ‘segment’ is referring e.g., about 1 to about 5 cm (in length, 1) inflatable sleeves located in the perimeter of the hand; about 0.1 to about 2 cm; about 4 to about 25 cm etc; wherein segments width can vary to suit the arm and palm width (W).

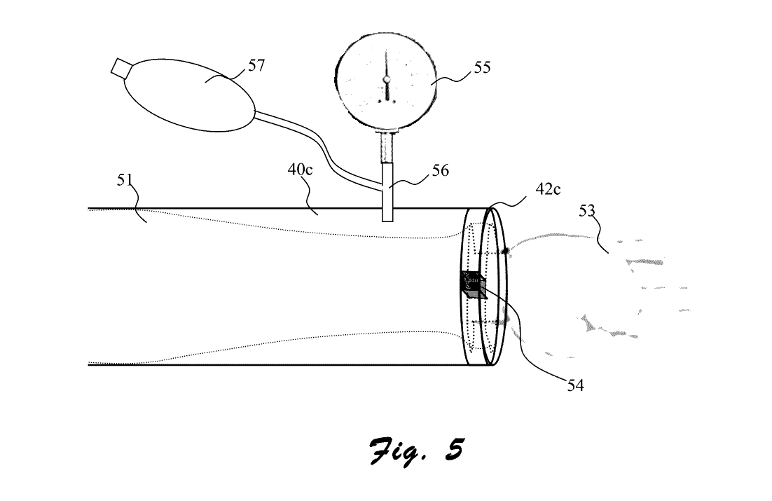

Reference is now made to FIG. 4a, schematically illustrating an out-of-scale manner the aforesaid set of three human right hands (1a-c), accommodated within a size-adjustable sleeve (40a-c, respectively). This single segment sleeve is telescopically reciprocated from a short to long configuration by inflating and deflating a party-horn like arrangement having a proximal portion (41) and an opposite distal portion (42), reversibly altering its length from a narrow configuration (40a) with a relatively long portion of tightly folded sleeve (42a); a medium length 40b with medium size remaining deflated sleeve portion (42b) and long sleeve (40c) having no residual inflated portion (42c). Width, like length can be altered from a narrow to relatively wide dimension.

It is in the scope of the invention where other elongation mechanisms are applied as a suitable embodiment of the technology, such as sleeves sheathing and unsheathing, tubular member's sleeve and unsleeve telescopic elongation, sleeve-in-sleeve “trombone”-like arrangement etc.

Reference is now made to FIG. 5, schematically illustrating an out-of-scale manner a size-adjustable sleeve of the present invention (here in its full elongated configuration, 40c). the sleeve is inflated by a pump (57) in a fluid communication (56) with at least one portion or segment of the sleeve for a defined specific pressure (55), e.g., a pressure between about 30 to about 60 mm Hg.

According to an embodiment of the invention, a sensor (54) or alternatively a plurality of sensors are incorporated with, to or in the sleeve, and/or comprised or communicated with both the body organ to be scanned (51) and the sleeve. The sensor is configured to one or more of the following (a) ensure the positioning or configure of the sleeve in respect to the body organ to be scanned, (b) detect the applied pressure (55), (c) feedbackly adjusting the sleeve configuration, pressure etc., and/or scan parameters, including organ's movement, vibration, temperature etc., (d) continuously or batch-wisely detect physiological parameters related with the subject to be analyze, such as heart pulse and parameters thereof, oximetry and oxygen saturation etc. thereby regulate (i) sleeve pressure, configuration or location, and/or (ii) scanning procedure.

It is in the scope of the invention wherein the sensor(s) is/are a pressure sensor, conductivity sensor, 1D, 2D or 3D acidometer sensor, acoustic sensor, a thermometer, an optical sensor, a pulseoximetry sensor, an oxygen saturation sensor or the like.

It is well in the scope of the invention that scanning operator and/or patient himself can regulate said applied pressure. Thus, pressure is applied either in a manual manner of automatically as said above.

It is in the scope of the invention wherein the sensor(s) are communicated with the sounding systems and subsystems in a MRI safe wireless communication or by means of commercially available MRI-safe wires.

It is in the scope of the invention wherein the conductive material is a copper net or an electrically conductive layer thereof. It is further in the scope of the invention wherein the electrically conductive materials are selected, in a non limiting manner from a group consisting of non-magnetic, electrical conductor, such as copper, silver, or aluminum, conductive polymers, such as polyacetylene, polyaniline, polypyrrole, polythiophene, and polyphenylene; synthetic or non-synthetic fibers which comprise carbon derivatives such as carbon nanotubes, carbon black, graphite; fabrics incorporated with inorganic conductive materials, tin oxide, copper sulfide, nickel graphite and any combination thereof.

It is further in the scope of the invention wherein the electrically conductive material or layer thereof is in the form of a mesh or a knitted fabric such that said mesh size is significantly smaller than the wavelengths to be reflected. It is further in the scope of the invention wherein the protective member comprises at least one of the following: layer of fabric or textile such as cotton, polyester, nylon and silk, high-performance fibers including Kevlar®, Nomex®, Technora® and Vectran®, for close-fitting on said cylindrical organ. It is further in the scope of the invention wherein two or more layers of fabric are in fluid connection to a fluid source and pump, positioned to entrap a fluid therein, thereby providing expansion of the fabric sleeve to fit dimensions of said organ in a tourniquet-like manner It is further in the scope of the invention wherein the protecting member is designed to provide an effective insulation for non imaged body extremities. It is further in the scope of the invention wherein the electrically conductive loop is connected to the protective earthing terminal in a way that they cannot be separated without the aid of a tool, according to IEC 60601-1-2 Medical Electrical Equipment standards. It is further in the scope of the invention wherein RF field effects are caused by one or more of the following: external RF such as radio waves from the vicinity of said MRD suite and RF electromagnetic radiation produced by nuclear relaxation inside the subject or internal RF from the radio frequency transmission system of said MRD, or any combination thereof. It is further in the scope of the invention wherein the protecting member provides an effective Faraday shielding thus performing as a barrier to prevent entry of external RF from the environment of the MRD to the MRD's bore and vice versa.

In one example, yet still in a non limiting manner, a protective member as shown in any of the embodiments illustrated and disclosed above, effectively reduces internal RF field effects within the MRD's bore at frequencies of about 100 MHz, and eliminate ohmic tissue heating, heating of conductors, and interference with patient monitoring equipment; and vice versa, the protecting gear reduces external RF field effects of the environment of the MRD to penetrate the inner bore and VOI of the MRD.

In another example, provided herein still in a non limiting manner, a protective member as shown in any of the embodiments illustrated and disclosed above, effectively eliminates tissue heating due to RF magnetic fields during MR scanning, and limited temperature rise in excess of 1° C. and localized heating up to 38° C. in the head, 39° C. in the trunk, and 40° C. in the extremities.

In another example, provided herein still in a non limiting manner, a protective member as shown in any of the embodiments illustrated and disclosed above, effectively maintains SAR recommended levels below values as follows: 4 Watt/kg averaged over the whole body for any 15-minute period (1.5 Watt/kg if patient is thermally compromised, as a function of room temperature and humidity), 3.2 Watt/kg averaged over the head for any 10-minute period, and 8 W/Kg in any 1 cc of tissue in head averaged over 5 minutes.

In another example, provided herein still in a non limiting manner, a protective member as shown in any of the embodiments illustrated and disclosed above, effectively maintain magnetic fringe field at the entrance to MRI suite to be equal to or less than 5 Gauss, referred to as the 5-Gauss line.

In another example, provided herein still in a non limiting manner, a protective member as shown in any of the embodiments illustrated and disclosed above, effectively maintain prevents temperature rise due to RF heating during MR scanning beyond said limit of 40° C. in said body extremity.

In another example, provided herein still in a non limiting manner, a protective member as shown in any of the embodiments illustrated and disclosed above, is adapted to fit one or more of said body extremities such as a toe, a finger, a wrist, an elbow, an ankle, a knee, a head, non- body extremities such as the abdomen, belly etc, and any combination thereof.

In another example, provided herein still in a non limiting manner, a protective member as shown in any of the embodiments illustrated and disclosed above, is adapted to provide separation means of skin-to-skin contact of body parts selected from the group consisting inner thigh-to-thigh, calf-to-calf, hand-to-hand, hand-to-body, ankle-to-ankle contact, thereby preventing formation of conductive loops through part of the body.

In another example, provided herein still in a non limiting manner, a protective member as shown in any of the embodiments illustrated and disclosed above, is adapted to provide means for preventing placement of said body extremity against an RF transmitting coil surface.

In another example, provided herein still in a non limiting manner, a protective member as shown in any of the embodiments illustrated and disclosed above, is adapted to provide SAR conforming to NEMA MS-8-2006 for MRI Systems.

In another example, provided herein still in a non limiting manner, a protective member as shown in any of the embodiments illustrated and disclosed above, is adapted to fit an orifice of a body therein.

In another example, provided herein still in a non limiting manner, a protective member as shown in any of the embodiments illustrated and disclosed above, comprises as follows: A sleeve-like organ-accommodating article of manufacture comprising a fabric sleeve (1); an intermediate conducting layer (2); an outer dielectric layer (3) an outer conducting strip (4); wherein said fabric sleeve is adjusted to accommodate a body extremity, said intermediate conducting layer is placed/sewn/surrounds said fabric sleeve, said outer dielectric layer is placed/sewn/surrounds said intermediate conducting layer, and said outer conducting strip is fastened to the outer dielectric layer with at least one contact with said intermediate conducting layer and at least one contact to an external earthing system (5).

In another example, provided herein still in a non limiting manner, a protective member as shown in any of the embodiments illustrated and disclosed above, applies with Electrical & Mechanical Safety (IEC 60601-1) General requirements for basic safety and essential performance.

In another example, provided herein still in a non limiting manner, a protective member as shown in any of the embodiments illustrated and disclosed above, applies with Electromagnetic Compatibility (IEC 60601-1-2) General requirements for basic safety and essential performance—Collateral standard: Electromagnetic compatibility—Requirements and tests.

In another example, provided herein still in a non limiting manner, a protective member as shown in any of the embodiments illustrated and disclosed above applies with IEC 60601-2-33 Medical electrical equipment—Part 2-33: Particular requirements for the basic safety and essential performance of magnetic resonance equipment for medical diagnostic (2007 (Second Edition)+A1:2005+A2:2007).

In another example, provided herein still in a non limiting manner, a protective member as shown in any of the embodiments illustrated and disclosed above, applies with Acoustic Noise Measurement Procedure for Diagnosing Magnetic Resonance Imaging Devices

In another example, provided herein still in a non limiting manner, a protective member as shown in any of the embodiments illustrated and disclosed above, applies with NEMA MS 8 (2006) Characterization of the Specific Absorption Rate (SAR) for MRI Systems.

In another example, provided herein still in a non limiting manner, a protective member as shown in any of the embodiments illustrated and disclosed above, applies with NEMA MS 10-2006 Determination of Local Specific Absorption Rate (SAR) in Diagnostic Magnetic Resonance Imaging

In another example, provided herein still in a non limiting manner, a protective member as shown in any of the embodiments illustrated and disclosed above, applies with NEMA MS 11-2006 Determination of Gradient-Induced Electric Fields in Diagnostic Magnetic Resonance Imaging

In another example, provided herein still in a non limiting manner, a protective member as shown in any of the embodiments illustrated and disclosed above, applies with IEC 60601-2-33 Medical electrical equipment—Part 2-33: Particular requirements for the basic safety and essential performance of magnetic resonance equipment for medical diagnostic (2007 (Second Edition)+A1:2005+A2:2007).

What is claimed is:

1. In an open bore MRD having a volume of interest for imaging a body portion and an inlet aperture for inserting said body portion within said bore, an electrically earthed size adjustable-size protecting and/or immobilizing sleeve.

2. An MRI-proof adjustable-size protecting sleeve characterized by a telescopic elongation mechanism.

3. An MRI-proof adjustable-size protecting sleeve comprising a pressure applying mechanism.

4. A method of RF protecting and/or immobilizing a body organ to be scanned by an open bore MRD having a volume of interest for imaging a body portion and an inlet aperture for inserting said body portion within said bore, comprising step of size adjusting said a sleeve such as said sleeve is set tightly at least a portion of the organ to be scanned.

5. A method of RF protecting and/or immobilizing a body organ to be scanned by an open bore MRD, comprising step of providing an MRI-proof adjustable-size protecting sleeve characterized by a telescopic elongation mechanism.

6. A method of RF protecting and/or immobilizing a body organ to be scanned by an open bore MRD, comprising step of providing an inflatable MRI-proof adjustable-size protecting sleeve.

7. A method of RF protecting and/or immobilizing a body organ to be scanned by an open bore MRD, comprising step of providing an MRI-proof adjustable-size protecting sleeve comprising pressure applying mechanism.

8. A method of RF protecting and/or immobilizing a body organ to be scanned by an open bore MRD, comprising step of providing an MRI-proof adjustable protecting sleeve comprising one more sensors.

9. The method of claim 8, comprising a step of inflating or deflating said sleeve.

10. The method of claim 8, comprising a step of applying, by means of said sleeve, a pressure upon said organ to be scanned.

11. The method of claim 8, comprising step of regulating said pressure by either manual or automatic feed backed mechanism.
